(19) United States
(12) Patent Application Publication
(10) Pub. No.: US 2015/0344959 A1
(43) Pub. Date: Dec. 3, 2015

(54) COMPOSITIONS AND METHODS FOR GENOTYPING CANINES

(71) Applicant: CORNELL UNIVERSITY, Ithaca, NY (US)

(72) Inventors: Adam Boyko, Ithaca, NY (US); Jessica Hayward, Freeville, NY (US)

(21) Appl. No.: 14/760,070

(22) PCT Filed: Jan. 14, 2014
(86) PCT No.: PCT/US14/11448

§ 371 Date: Jul. 9, 2015

Related U.S. Application Data
(60) Provisional application No. 61/752,215, filed on Jan. 14, 2013.

(51) Int. Cl. C12Q 1/68

(52) U.S. Cl. CPC C12Q 1/6883; C12Q 2600/156; C12Q 2600/124

(57) ABSTRACT

Provided are compositions and methods for use in genotyping canines. The compositions and methods are useful for determining canine genotypes related to certain canine phenotypes, including shedding and/or long hair, body size, and ear type. The compositions and methods involve determining single nucleotide polymorphisms in biological samples obtained from canines.

### CROSS REFERENCE TO RELATED APPLICATIONS

This application claims priority to U.S. Provisional application No. 61/752,215, filed on Jan. 14, 2013, the disclosure of which is incorporated herein by reference.

### FIELD OF THE INVENTION

The present disclosure relates generally to canine genetics and more specifically to compositions and methods for determining genetic features related to canine phenotypic traits.

### BACKGROUND OF THE INVENTION

While many studies on canine genetics have identified markers associated with a wide variety of disease and phenotypes, there remains an ongoing need to identify additional markers for use in determining genotypes of individual canines and using those markers to assist with canine breeding. The present disclosure meets these and other needs.

### SUMMARY OF THE INVENTION

In embodiments the instant disclosure provides compositions and methods for determining canine genotypes for use in predicting certain canine phenotypes, including shedding/long hair, body size (i.e., height and weight), or ear type. The disclosure includes determining single nucleotide polymorphisms (SNPs) in biological samples obtained from canines. The SNPs are useful for determining whether the canine genome included a chromosome that comprised any individual or any combination of the SNPs described herein.

In an embodiment the method comprises determining in vitro in a biological sample comprising canine DNA a single nucleotide polymorphism (SNP) marker indicative of a phenotype of high shedding or low shedding. The method comprises testing DNA in the sample to determine the nucleotide at position 61 in SEQ ID NO:1 (which is a segment of MCR5), wherein determining a C at position 61 indicates the SNP is from a chromosome from a canine having the phenotype of high shedding, and determining a T indicates the SNP in the biological sample is from a chromosome from a canine having the phenotype of low shedding.

In embodiments the method comprises contacting the canine DNA from the biological sample with a first and second DNA probe. The first probe is specific for or can be extended to include the SNP that is from the chromosome from the canine that is a high shedder. The second probe can be specific for or can be extended to include the SNP that is from the chromosome from the canine that is a low shedder. The method includes detecting hybridization or extension of only the first probe, or detecting hybridization or extension of only the second probe, or detecting hybridization or extension of the first and second probes, thereby determining heterozyosity or homozygosity for either allele.

The probes can be attached to a substrate and can be members of a plurality of distinct DNA probes in an array, such as on a chip. The method includes determining any of the SNPs by amplifying a segment of canine genomic DNA and determining the sequence of the SNP, and optionally determining sequences which flank the SNP.

Another aspect of the disclosure includes a method for determining in vitro in a biological sample comprising canine DNA a marker indicative of canine body size. The method comprises testing DNA in the biological sample to determine a SNP selected from the nucleotide at position 61 in SEQ ID NO:2 (which is near FGFR3), wherein determining a C at position 61 indicates the SNP in the biological sample is from a chromosome from a canine having a small body size, and determining a T indicates the SNP in the biological sample is from a chromosome from a canine having a large body size. In another embodiment, the method involves testing the nucleotide at position 61 in SEQ ID NO:4 (which is a segment of SMOC2), wherein determining an A at position 61 indicates the SNP in the biological sample is from a chromosome from a canine having a small body size, and determining a G indicates the SNP in the biological sample is from a chromosome from a canine having a large body size. In another embodiment the method comprises determining the nucleotide at position 61 in SEQ ID NO:10 (which is a segment of TBX19), wherein determining a G at position 61 indicates the SNP in the biological sample is from a chromosome from a canine having a small body size, and determining an A indicates the SNP in the biological sample is from a chromosome from a canine having a large body size. In another embodiment the method includes determining the nucleotide at position 61 in SEQ ID NO:15 (which is near OGFRL1), wherein determining a C at position 61 indicates the SNP in the biological sample is from a chromosome from a canine having a small body size, and determining an A indicates the SNP in the biological sample is from a chromosome from a canine having a large body size. In another embodiment the method includes determining the SNP at position 61 in SEQ ID NO:18 (which is near MITF), wherein determining an A at position 61 indicates the SNP in the biological sample is from a chromosome from a canine having a small body size, and determining a C indicates the SNP in the biological sample is from a chromosome from a canine having a large body size; or determining a combination of said SNPs.

In embodiments the method of testing to determine body size comprises contacting the canine DNA with a first and second DNA probe wherein the first probe is specific for or can be extended to include a SNP that indicates the SNP in the sample is from a chromosome from a canine with a small body size, wherein the second probe is specific for or can be extended to include a SNP that indicates the SNP in the sample is from a chromosome from a canine with a large body size. Use of such probes facilitates detecting hybridization or extension of only the first probe, or detecting hybridization or extension of only the second probe, or detecting hybridization or extension of the first and second probes, thereby determining zygosity for the SNP(s) in the sample.

In another aspect the disclosure includes a method for determining in vitro in a biological sample comprising canine DNA a SNP marker indicative of a phenotype of floppy ears, the method comprising testing the DNA to determine the nucleotide at position 61 in SEQ ID NO:3 (which is a segment of MSRB3), wherein a G at position 61 indicates the SNP in the biological sample is from a chromosome from a canine that had floppy ears, and determining an A at position 61 indicates that the SNP in the biological sample is from a chromosome from a canine that did not have floppy ears. First and second probes as outlined above can be used to determine the SNP, provided they are modified for recognition of the floppy ear SNPs described herein.

In an embodiment, the disclosure includes a method for determining that a biological sample comprises DNA that was obtained from a canine. The method comprises testing DNA in the sample to determine the presence or absence of any of the SNPs and/or sequences disclosed herein, wherein determining the SNPs and/or sequences indicates the sample comprises canine DNA that was obtained from a canine having a genome which comprises, for example, SEQ ID NO:1, wherein the nucleotide at position 61 is a T, or determining that the biological sample contains DNA that was obtained from a canine having a genome which comprises SEQ ID NO:1, wherein the nucleotide at position 61 is a C, the method comprising testing the DNA to determine the presence or absence of SEQ ID NO:1 and determining the nucleotide at position 61 in SEQ ID NO:1.

In embodiments the disclosure includes articles of manufacture for use in determining a phenotype for a canine, the article comprising a first and second DNA probes that are specific for or can be extended to include any of the SNPs disclosed herein.

### BRIEF DESCRIPTION OF THE FIGURES

FIG. 1 shows a Manhattan and QQ plot male body size (weight) for 152 breeds (1,812 individual dogs genotyped). For each breed, male average weight was transformed as ŵ0.38 based on Box-Cox analysis. 18 genomic regions reach genome-wide significance (FDR<0.01), six of which have not been previously associated with body size in dogs.

FIG. 2 shows a Manhattan and QQ plot of body size (height) for 152 breeds (1,812 individual dogs genotyped).

FIG. 3 shows a Manhattan and QQ plot of average shedding based on 125 breeds (81 light, 27 average, and 17 heavy shedding breeds; 1,477 individuals total).

FIG. 4 shows a Manhattan plot of earflop (floppy vs not) and QQ plot of floppy (drop) ears as a breed-fixed trait (76 drop-eared breeds vs 71 other breeds; N=903 and 760 individuals, respectively). A single QTL region localizes to CFA10 between 8.013-8.135 Mb which encompasses a single gene region, the last two exons of MSRB3. This region contains two nonsynonymous mutations, including a G->A transition at position 8,037,693 inducing a Gly->Ser amino acid change (G198S).

FIG. 5 shows a Manhattan and QQ plot of fur length scored as a breed-fixed quantitative trait scored as 1 (short) to 5 (long) (1,724 individuals total). Three significant QTLs are apparent: MCSR (FIG. 3), and known variants in RSPO2 and FGF5.

FIG. 6 is a chart providing sequences of probes for use in determining markers, the marker name and the sequence identifiers.

FIG. 7 is a chart showing the chromosome where the locus and markers are found, the gene, the effect, a marker identification number, and designations of the reference and altered alleles and their correlation with body size. The SNPs are either within or near the genes.

### DETAILED DESCRIPTION OF THE INVENTION

The present disclosure provides compositions and methods that are useful for, among other purposes, determining canine genotypes for use in predicting certain canine phenotypes. In general, in contrast to previous breed-mapping studies, our approach included individual measurements for most dogs. Pooled genome-wide association studies (GWAS) across breeds using individual versus breed-average measures yielded similar results, and GWAS within breeds enabled identification of additional loci affecting traits as described further herein.

In embodiments, methods of this disclosure comprises testing a biological sample to determine that it contains canine DNA, and/or to determine if the canine DNA comprises one or more markers described herein. In various embodiments the method generally comprises testing a sample comprising nucleic acids from a canine for any one or any combination of the allelic variants that are described in this disclosure. In embodiments the allelic variants comprise single nucleotide polymorphisms (SNPs). In embodiments, determining the nucleotide at the SNP location signifies the presence of an allele that is associated with a phenotype. In embodiments the disclosure is useful for testing a sample from a canine to determine the nucleotide at a SNP location that is indicative of the canine's phenotype for shedding/long hair, body size (i.e., height and weight), or ear type. Thus in embodiments, testing the biological sample and determining a marker indicates the sample was obtained from a canine that has a phenotype of high shedding or low shedding, or large size or small size, or as having floppy ear or not. The disclosure includes concurrently determining combinations of the markers. The methods are not limited to any particular breed, gender or age of canine.

The biological sample tested according to the invention can be any biological sample that contains or would be suspected to contain nucleic acids. Thus, any sample comprising nucleated cells can be used as source of nucleic acids. In non-limiting embodiments, the sample can be a tissue sample, a hair sample, or a sample of a biological liquid, including but not necessarily limited to saliva, serum, blood, or urine. In embodiments the sample is a saliva swab or blood sample. In certain approaches, the sample is obtained from the canine subject and used directly in determining the SNP, meaning determining the specific nucleotide at the SNP location. In other embodiments, the biological sample is obtained and subjected to a processing step before the biological sample is used in testing for the SNP or other mutation. In some examples, the processing step can be carried out to isolate, and/or purify the polynucleotides for testing.

The disclosure includes testing nucleic acid using any suitable technique. In certain embodiments the method involves separating canine DNA and/or RNA from a biological sample, and mixing the separated nucleic acids in a reaction container with non-naturally occurring reagents, including but not necessarily limited to synthetic oligonucleotide primers and/or probes described herein. Additional reagents that can be added into the reaction mixture include but are not limited to salts, buffers and the like, a recombinant prokaryotic or bacteriophage DNA polymerase, free nucleoside triphosphates, etc. In this regard, genomic DNA and/or RNA can be tested directly or may be amplified enzymatically in vitro by, for example, use of the polymerase chain reaction (PCR), or any other in vitro amplification methods.

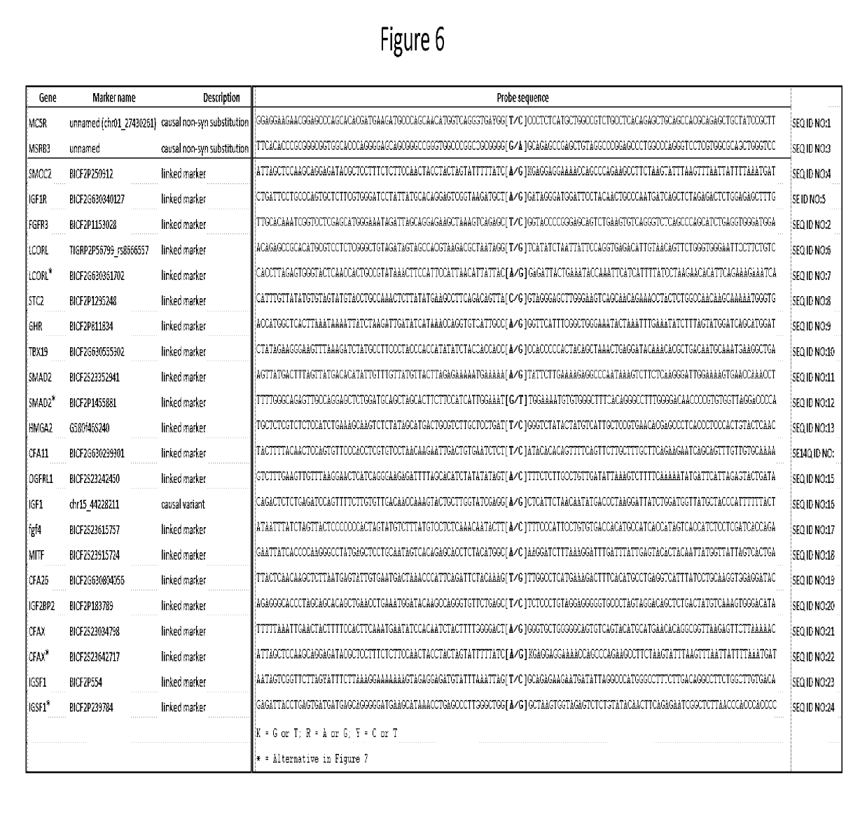

For amplification and/or primer extension and/or sequencing reactions and/or hybridization approaches, probes can be designed which hybridize to genomic DNA (or mRNA where the informative nucleotides are transcribed), and used to obtain nucleic acid amplification products (i.e., amplicons comprising amplified genomic DNA and/or amplified cDNA). Those skilled in the art will recognize how to design suitable primers and perform amplification and/or hybridization reactions in order to carry out various embodiments of the method. In general, the probes should be long enough to be useful in amplification or DNA sequencing reactions, and generally are at least 12 bases in length, but probes as short as 8 bases can be used depending on reaction conditions. In embodiments, the probes can comprise or consist of between 8 and 170 nucleotides, inclusive, and including all integers and ranges there between. In embodiments, the probes can be at least 121 nucleotides in length, or can consist of 121 nucleotides. In embodiments, the probes comprise between at least 10 and up to 61 contiguous nucleotides of the sequences presented herein, provided the contiguous sequence includes the nucleotide at the SNP location, which is the location of the variable nucleotide for each of the representative probes presented in FIG. 6. The probes used for detecting canine nucleic acids can comprise modifications, such as being conjugated to one or more detectable labels, such as fluorophores, metals or chemiluminescent moieties. In embodiments, the probes can be fixed to a solid substrate, such as glass or a silicon chip, or microscopic beads made of any suitable material, such as glass or polystyrene. The probes can be covalently bound to the surface after the probes are synthesized, or the probes can be directly synthesized on the solid substrate.

In embodiments, canine polynucleotides are tested using DNA microarrays comprising polynucleotide probes, wherein the probes are designed to discriminate SNPs that are associated with the canine traits as further described herein. In embodiments, for any one or any combination of the markers set forth in this disclosure, a DNA array or any chip or bead format for testing a plurality of polynucleotides can be provided. Various reagents, devices and procedures which comprise polynucleotide arrays and are used for analyzing nucleic acid samples are known in the art, are commercially available and can be adapted for use with the present disclosure. For instance, devices and services sold under the trade names ILLUMINA and AFFYMETRIX can be adapted to test biological samples obtained or derived from canines for any one or any combination of the markers discussed herein, given the benefit of this disclosure.

In general, in certain embodiments that can comprise arrays, for any particular marker, at least two probes are attached to a substrate. The at least two probes can differ from each other such that one probe will only hybridize to a genomic DNA segment that comprises a reference allele, and the other will only hybridize to a genomic DNA segment which comprises a variant allele. In embodiments two probes for testing a particular marker can differ from one another only at the SNP location, and thus can differ in only a single nucleotide. A “reference” allele means the particular nucleotide at a particular chromosomal position that is numbered according to the publicly available “CanFam” canine genome sequence assembly. Unless specified or would otherwise be apparent to those skilled in the art, chromosomal nomenclature and nucleotide numbering in this disclosure is as set forth in CanFam3.1, released September 2011, accessible at: ftp://ftp.broadinstitute.org/distribution/assemblies/mammals/dog/canFam3.1/. As will be apparent from this disclosure, any particular trait can be associated with a reference allele, or with a variant allele. In certain cases, the allele associated with a trait may also be partially or fully causative of that trait.

In embodiments, any of the DNA sequences presented herein and any combination of them can be used in a DNA array on a chip. “DNA array” and “chip” are not intended to be limited to any particular configuration, and include all devices, articles of manufacture and processes that are used for concurrent testing of a plurality of distinct nucleic acids to determine multiple distinct SNPs present in the distinct polynucleotides. In embodiments any combination of the probes disclosed herein can be the only probes in an array, or there can be other probes on the array, including probes that may be designed to test for other markers that are related to canine phenotypes, and/or markers that are not necessarily informative with respect to a phenotype, but may be informative for, for instance, a disease trait. Probes directed to DNA from non-canine animals may also be included. In embodiments, the probes are selected from probes having the sequences presented in FIG. 6. In this regard, it will be recognized that each DNA sequence presented herein includes its reverse complement, which can also be used to interrogate any SNP that is described herein. Likewise, every SNP in this disclosure includes the reverse complement of that SNP. It is accordingly considered that determining any particular SNP includes a determination of the complement of that SNP. To the extent any of the markers described herein are transcribed into RNA, the disclosure includes optionally testing the mRNA and/or cDNA made from the mRNA.

In an embodiment, the disclosure includes an article of manufacture for use in determining a phenotype for a canine. The article comprises combinations of probes, wherein the probes are attached to a substrate. In embodiments, the article of manufacture comprises at least two probes directed to one of the markers discussed in this disclosure, wherein a first probe is directed to one allele of the marker and the second probe is directed to a second allele of the marker. As a non-limiting illustration that will generally apply to all of the markers and probes described herein, in one embodiment, an article of manufacture comprises a first and second DNA probe, wherein the first probe is specific for a segment of SEQ ID NO:1 that comprises nucleotide 61 that is a C, and wherein the second probe is specific for a segment of SEQ ID NO:1 that comprises nucleotide 61 that is a T, wherein the first and second probes are attached to a substrate. In embodiments the article of manufacture comprises a DNA chip.

Specifically, SEQ ID NO:1 is:

GGAGGAAGAACGGAGCCCAGCACACGATGAAGATGCCCAGCAACATGGTC
AGGGTGAYGG[T/C]CCCTCTCATGCTGGCCGTCTGCCTCACAGAGCTGC
AGCCACGCAGAGCTGCTATCCGCTT.

SEQ ID NO:1 consists of 161 nucleotides because only a T or C (or A or G in the reverse complement) will be present at position 61. According to convention, SEQ ID NO:1 included with the sequence listing submitted with this disclosure designates position 61 as “n” which, for SEQ ID NO:1 means that “n” is T or C (including the complementary nucleotides if the reverse strand sequence is used). The same convention is followed for the other sequences provided in FIG. 6 per their respective SNPs. Additionally, in certain sequences in FIG. 6, K, R and Y are included. K signifies G or T; R signifies A or G and Y signifies C or T. Thus, the Y at position 58 in SEQ ID NO:1 can be C or T. These symbols are also given as “n” in the sequence listing accompanying this disclosure. In embodiments, for testing canine polynucleotides for predicting a shedding phenotype, two probes which can differ from one another only by the nucleotide at position 61 in SEQ ID NO:1 are attached to a substrate, and/or are used in amplification reactions. The probes can comprise or consist of any fragment of SEQ ID NO:1 or its reverse complement, so long as the fragments are of adequate length to specifically recognize test canine genomic DNA and they include or can be extended to include the nucleotide at the SNP position. Thus, in embodiments, one probe will have C at the SNP position and the other will have T at the SNP position (or their complement(s) as described above). Either or both probes can have the SNP nucleotide at or near its 3′ terminal position, or the SNP nucleotide can be present elsewhere in the sequence. Alternatively, the probe may not include the SNP, but is configured such that its 3′ end will be near or will terminate with the nucleotide immediately preceding the SNP location such that the presence or absence of the SNP will dictate extension of the probe such that extension can determine either SNP nucleotide. If desired, primer-extended DNA can be amplified and/or sequenced to determine the SNP.

The test may be configured such that a test canine nucleic acid will only detectably hybridize to the probe that contains its complementary SNP, or the probe will only be extended based on the presence or absence of either SNP. Hybridization of the test nucleic acid facilitates generation of a detectable signal using, for example, primer extension reactions with detectably labeled moieties, or other techniques that are known and used in the art. Thus, to provide one non-limiting illustration, when testing a canine DNA sample that is homozygous for T at the SNP position in SEQ ID NO:1, a meaningful signal will only be produced from hybridization with and/or extension from to its cognate probe, and there will be no meaningful signal from the probe which includes the other allele because the test DNA will not hybridize to it and/or extend from it such that a meaningful signal is produced. For a heterozygote, only one allele will hybridize to and/or be extended from the probe, which depending on the system used, can result in approximately half the signal intensity of a homozygote, where distinct signals are used for each allele. Those skilled in the art will recognize there are a variety of signals and techniques that can be used to discern zygosity in any particular sample for any particular allele and they can be adapted for use with the instant invention.

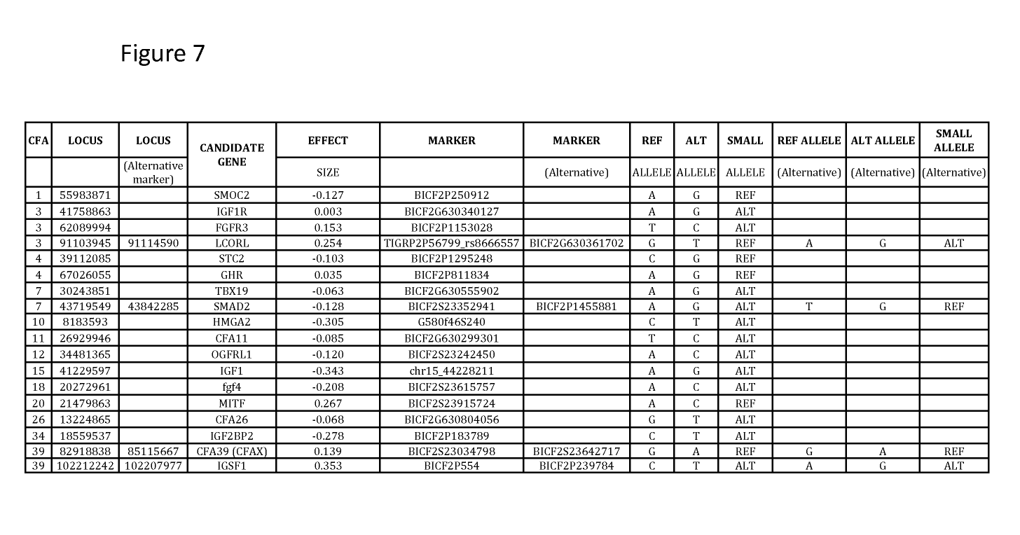

In the case of SEQ ID NO:1, T is the allele that co-segregates with low shedding, as well as short fur length, while C is the allele that co-segregates with more shedding, as well as longer fur. Thus, determining a C at position 61 in SEQ ID NO:1 in a canine DNA sample demonstrates that the sample was obtained from a canine which carries a trait to be a higher shedder with longer fur, while determining a T at that position determines the canine carries a trait to be a low shedder with shorter fur. Determining homozygosity for T determines the canine from which the sample was obtained is a lower shedder and has shorter fur than a heterozygote for that allele, or a homozygote for the C allele. Determining homozygosity for the C allele determines that the canine from which the sample was obtained is a higher shedder with longer fur than a heterozygote, or a homozygote for the T allele. This same rationale applies to all of the other markers, alleles and traits described in FIG. 6 and FIG. 7. Thus, in embodiments, testing the sample comprises determining whether the canine from which the sample was obtained is homozygous, heterozygous or hemizygous for any particular marker.

In another non-limiting embodiment, the method for any of the SNPs described herein may be adapted so that it is carried out using, for example, a process offered commercially under the trade name INFINIUM as INFINIUM II. In this embodiment, a portion DNA flanking the SNP location is used in a probe which is directed to a position up to but not including the nucleotide at the SNP of interest such that during single-base extension, only the base that is complementary to the SNP is extended. Thus, the result of the extension provides the genotype at that SNP. For instance, in the case of the MCR5 marker (chr01_{—}27430261) one probe sequence can comprise: CAGCTCTGCGTGGCTGCAGCTCTGTGAGGCAGACGGCCAGCATGAGAGGG (SEQ ID NO:25), which is the 5′ end of a DNA sequence that is complementary to: GGAGGAAGAACGGAGCCCAGCACACGATGAAGATGCCCAGCAACATGGTCAGG GTGAYGG[T/C]CCCTCTCATGCTGGCCGTCTGCCTCACAGAGCTGCAGCCACGCA GAGCTGCTATCCGCTT (SEQ ID NO:1). Thus, one probe to MCR5 can comprise or consist of the complementary sequence: AAGCGGATAGCAGCTCTGCGTGGCTGCAGCTCTGTGAGGCAGACGGCCAGCATG AGAGGG[A/G]CCRTCACCCTGACCATGTTGCTGGGCATCTTCATCGTGTGCTGGGC TCCGTTCTTCCTCC (SEQ ID NO:26), or a segment of it, which segment can comprise or consist of the sequence: CAGCTCTGCGTGGCTGCAGCTCTGTGAGGCAGACGGCCAGCATGAGAGGG (SEQ ID NO:25), wherein the 3′ terminal nucleotide, if extended, will encompass the SNP at position 61 of SEQ ID NO:1 on one strand of the double stranded DNA in the chromosome.

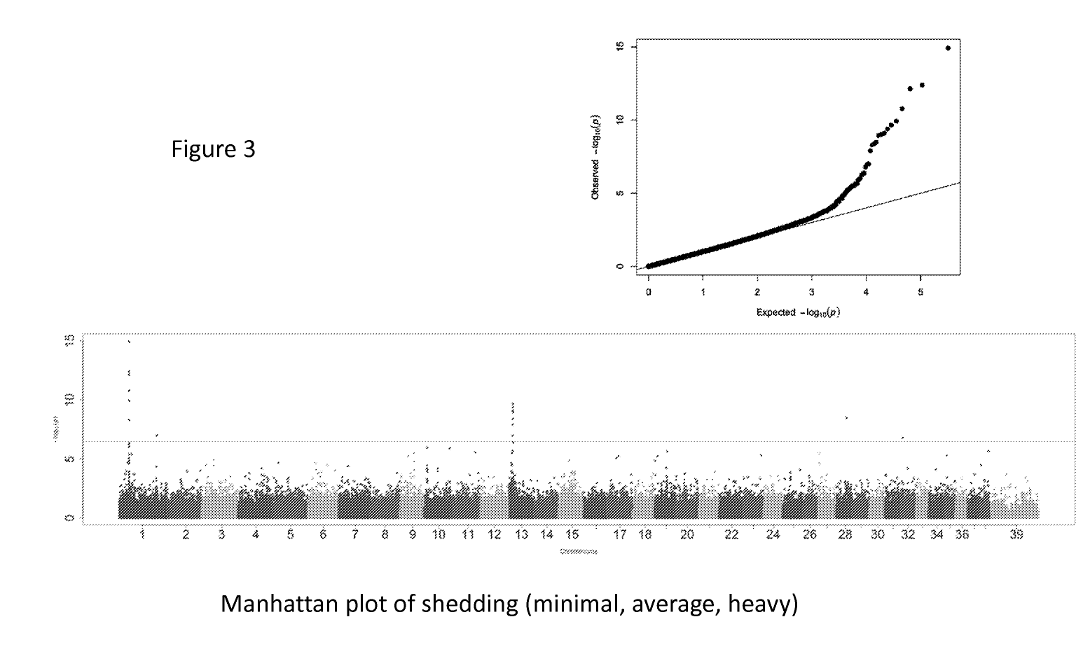

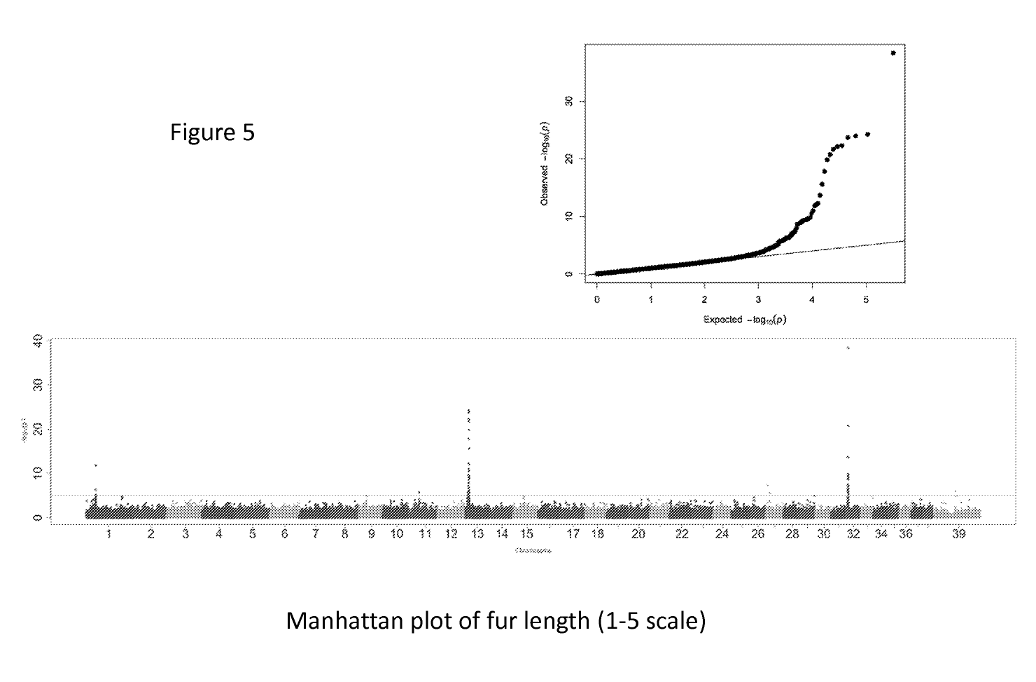

In a non-limiting illustration of an aspect of the invention using the shedding phenotype, we analyzed alleles associated with shedding phenotypes using 125 breeds (81 light, 27 average, and 17 heavy shedding breeds; 1,477 individuals total). The results are presented in FIG. 3 as a Manhattan and QQ plot. The allele with the highest association with shedding phenotype (the highest data point on chromosome 1) results in a missense (Ala->Tyr) mutation in MCSR. This is the SNP described in SEQ ID NO:1 with the T allele associated with the reference Ala residue and the C allele associated with the non-reference Tyr residue. Without intending to be constrained by theory, it is believed that MCSR may interact with a variant in RSPO2 previously associated with wire fur and furnishings to determine shedding propensity, but the present disclosure also provides for analyzing MCR5 alone to predict shedding phenotype, if desired. The MCSR variant, along with variants in FGF5, also is predictive of fur length (see FIG. 5, showing a Manhattan and QQ plot of fur length scored as a breed-fixed quantitative trait scored as 1 (short) to 5 (long) (1,724 individuals total). Three significant QTLs are apparent: MC5R, RSPO2, and FGF5. Thus, in embodiments, testing for shedding/fur length can comprise determining the SNPs for MCR5, RSPO2, and FGF5. In embodiments, the MC5R SNP is determined in the same array with one or both of the RSPO2 and FGF5 SNPs. In embodiments, determining shedding can be determined alone or in combination with fur length.

In one non-limiting example, the disclosure includes testing a sample of canine genomic DNA or DNA derived from it to determine whether or not the sample was obtained from a canine which exhibits high shedding or low shedding by exposing a segment of native or amplified genomic DNA to two probes. The two probes can comprise all or a portion of the sequence shown in the first row of FIG. 6, which is a probe directed to a segment of canine chromosome 1 in the MCSR gene region. A second probe can optionally be used to determine the genotype of the individual animal at the RSPO2 sequence previously identified (Cadieu et al 2009, Science, 326, Vol. 150. P149-150) and associated with wire-fur and/or furnished coat. Dogs homozygous for the non-reference RSPO2 sequence associated with wired-fur (Cadieu et al 2009) exhibit minimal fur shedding (e.g. poodles, cairn terriers, portuguese water dogs). Similarly, dogs with the reference T allele at this novel MCSR locus exhibit less shedding than dogs lacking the allele. For dogs containing one or more reference RSPO2 alleles, a probe based on the first row of FIG. 6 can determine the degree of shedding where dogs with no copies of the T reference allele would be the heaviest shedders (e.g. akitas, alaskan malamutes, shetland sheepdogs) and dogs with two copies of the T reference allele would be average shedders (e.g. cocker spaniels, english setters, pugs). More generally, the number of reference RSPO2 alleles and the number of non-reference MCSR alleles detected by the probe designed from the first row of FIG. 6 will be positively correlated with the amount of shedding of an individual animal, and can be used to qualitatively rank individual animals based on shedding propensity. This would provide for the first time the ability to predict shedding propensity in individual animals, including mixed-breed dogs, designer breed dogs, dogs from breeds where shedding propensity is variable, or dogs from breeds where shedding propensity has not be characterized. The RSPO2 genotype can be tested directly using the testing method described by Cadieu et al (2009), or it can be indirectly assayed on the same genotyping platform as the MCSR locus, for example, by including a probe for marker BICF2G630605341 such as GAGAGAGAGGTAACGGGATCCTGAAGCAGGGCTGAGGAATTAGCTATGCTTGTG AGGACA[C/T]CTGTCCTCACAGTCTGATTGCCTCAGCCCGACACTCAATCAGCCTG GAGGTGATGAGGCT (SEQ ID NO:27) where C denotes the non-reference RSPO2 allele.

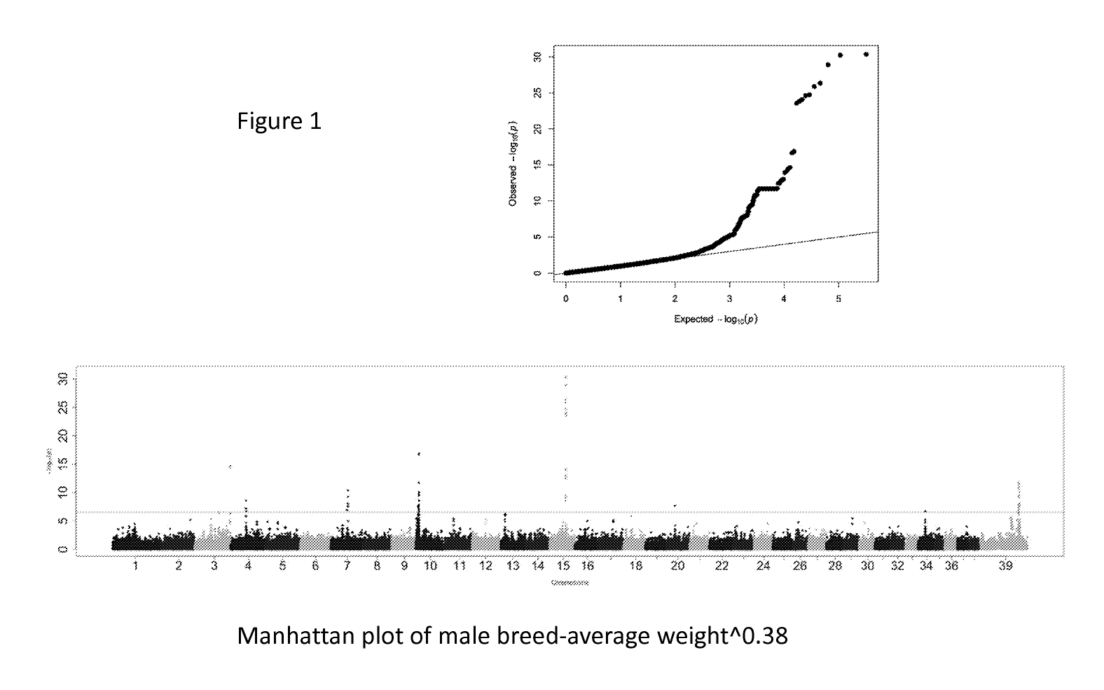

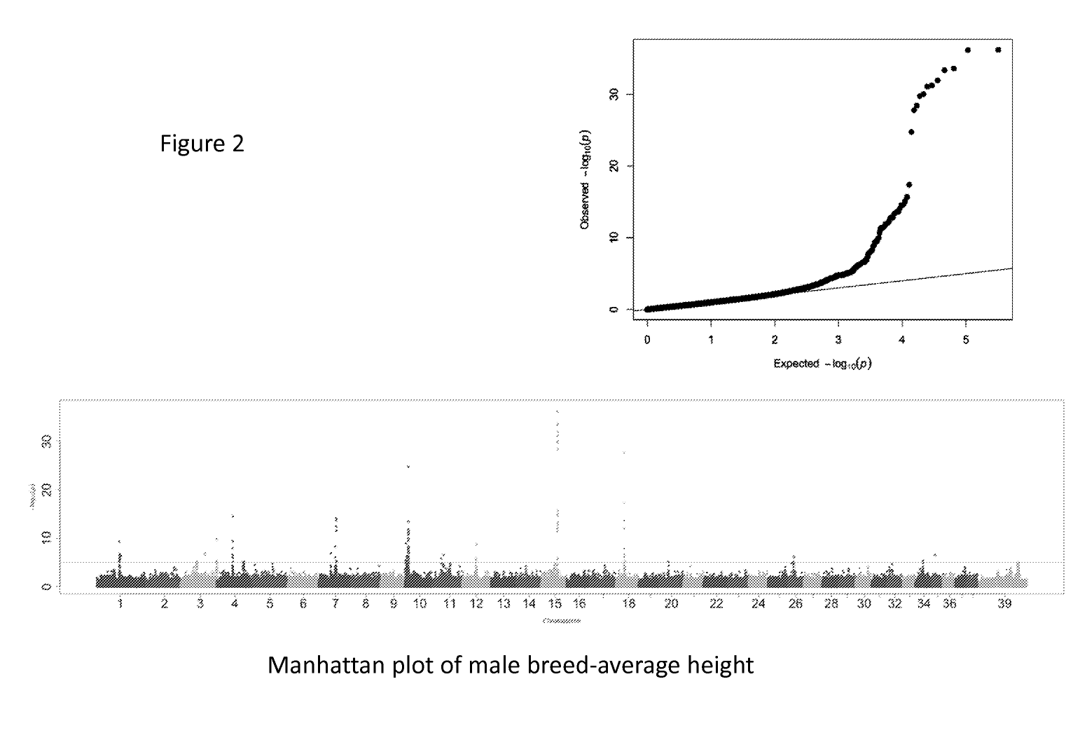

With respect to body weight, probes suitable for testing for this trait are described in FIG. 6. FIG. 7 shows which allele is the reference, which allele is the variant, and which allele correlates with the trait, where the trait is small body size. Conversely, the allele that does not correlate with small body size indicates larger body size. Body size takes into account height and weight and other physical dimensions (e.g. length, girth). Data obtained in analyzing height and weight markers described in FIGS. 6 and 7 are summarized in FIGS. 1 and 2. In particular, FIG. 1 provides a Manhattan and QQ plot of body size (weight) for 152 breeds (1,812 individual dogs genotyped). For each breed, male average weight was transformed as ŵ0.38 based on Box-Cox analysis. Eighteen genomic regions reach genome-wide significance (FDR<0.01). FIG. 2 provides Manhattan and QQ plot of body size (height) for 152 breeds (1,812 individual dogs genotyped). Markers in FIG. 6 not previously associated with body size include the markers described for OGFRL1, MITF, and FGFR3.

Any marker and any combination of markers and any combination of probes described in FIG. 6 for size can be used to test the canine nucleic acid sample. In an embodiment, at least one of the markers tested is the marker described for OGFRL1, MITF, or the FGFR3 gene in FIGS. 6 and 7. In an embodiment, at least one of the markers is the FGFR3 marker. Thus, in an embodiment, testing the canine nucleic acids for markers of body size can include testing for the SNP at position 61 in TTGCACAAATCGGTCCTCGAGCATGGGAAATAGATTAGCAGGAGAAGCTAAAGT CAGAGC[T/C]GGTACCCCGGGAGCAGTCTGAAGTGTCAGGGTCTCAGCCCAGCAT CTGAGGTGGGATGGA (SEQ ID NO:2) wherein the C is associated with small body size and the T is associated with large body size.

The same parameters for probe length and composition as described above for use with the shedding allele apply to probes for use with testing canine samples for body weight markers. Likewise, all of these parameters apply to probes for testing canine samples for ear type as describe below.

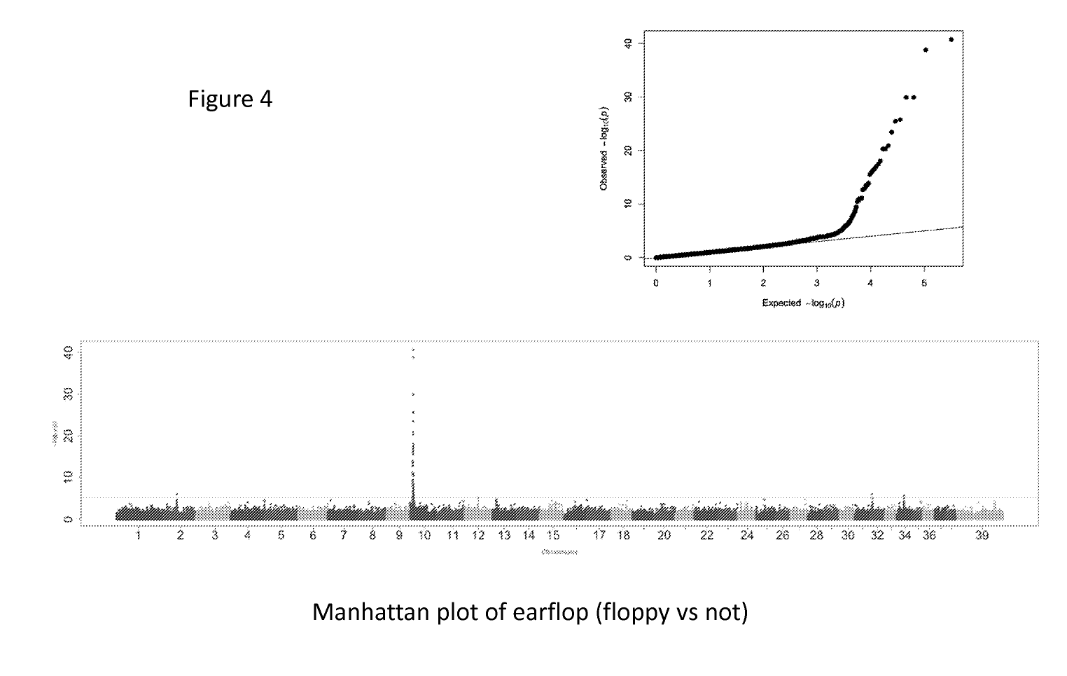

With respect to ear type, FIG. 6 identifies alleles in SEQ ID NO:3 at position 61. The G allele is associated with floppy ears, while the A allele is associated with non-floppy ears, such as folded/prick/rose. SEQ ID NO:3 is TTCACACCCGCGGGCGGTGGCACCCAGGGGAGCAGCGGGCCGGGTGGCCCGGCC GCGGGG[G/A]GCAGAGCCGAGCTGTAGGCCCGGAGCCCTGGCCCAGGGTCCTCG TGGCGCAGCTGGGTCC. Data obtained in the identification of this marker is summarized in the Manhattan and QQ plot of floppy (drop) ears shown in FIG. 4 as a breed-fixed trait (76 drop-eared breeds vs 71 other breeds; N=903 and 760 individuals, respectively). The SNP is in a single QTL region which localizes to CFA10 between 8.013-8.135 Mb, which encompasses a single gene region and includes the last two exons of MSRB3. This region contains two nonsynonymous mutations, including a G->A transition at position 8,037,693 inducing a Gly->Ser amino acid change. The floppy ear trait (also known as “drop ear” or “pendant ear” is an ear that folds and/or droops close to the head and is the standard/predominant ear conformation for Bloodhounds, Brittanies, Cocker Spaniels and numerous other breeds,

It will be recognized from the foregoing that in embodiments the disclosure provides for determining whether or not any particular canine is a trait carrier, and the likelihood and/or frequency that offspring from the canine will inherit the particular trait in question. Use of the compositions and methods of this disclosure will accordingly provide valuable information for dog breeders who can use the testing results to select breeding pairs to, for instance, produce litters which have increased or decreased shedding, body size (height and weight), or exhibit a floppy ear phenotype.

In various embodiments, the invention comprises fixing in a tangible medium the result obtained by testing for the canine DNA and determining any of the markers described herein. The tangible medium can be any type of tangible medium, such as any type of digital medium, including but not limited to a DVD, a CD-ROM, a portable flash memory device, or a printed or digitized report, etc., such as a spreadsheet or word processing document. The invention includes providing the tangible medium to an individual to assist with selection of canines for breeding.

While the invention has been described through specific embodiments, routine modifications will be apparent to those skilled in the art and such modifications are intended to be within the scope of the present invention.

What is claimed is:

1. A method for determining in vitro in a biological sample comprising canine DNA a single nucleotide polymorphism (SNP) marker indicative of a phenotype of high shedding or low shedding, the method comprising testing the DNA to determine the nucleotide at position 61 in SEQ ID NO:1, wherein determining a C at position 61 indicates the SNP is from a chromosome from a canine having the phenotype of high shedding, and determining a T indicates the SNP in the biological sample is from a chromosome from a canine having the phenotype of low shedding.

2. The method of claim 1, wherein the method comprises contacting the canine DNA from the biological sample with a first and second DNA probe, wherein the first probe is specific for or can be extended to include the SNP that is from the chromosome from the canine having the phenotype of high shedding, and wherein the second probe is specific for or can be extended to include the SNP that is from the chromosome from the canine having the phenotype of low shedding, and detecting hybridization or extension of only the first probe, or detecting hybridization or extension of only the second probe, or detecting hybridization or extension of the first and second probes.

3. The method of claim 2, wherein the first and second DNA probes are attached to a substrate.

4. The method of claim 3, wherein the first and second DNA probes are members of a plurality of distinct DNA probes in an array.

5. The method of claim 2, wherein the nucleotide at position 61 is determined to be a C.

6. The method of claim 2, wherein the nucleotide at position 61 is determined to be a T.

7. The method of claim 1, wherein the determining the nucleotide at position 61 in SEQ ID NO:1 comprises producing an amplicon comprising a segment of SEQ ID NO:1 that comprises nucleotide 61, and sequencing the amplicon to determine the nucleotide at position 61.

8. A method for determining in vitro in a biological sample comprising canine DNA a marker indicative of canine body size, the method comprising testing the DNA to determine one or more single nucleotide polymorphisms (SNPs) selected from the nucleotide at position 61 in SEQ ID NO:2, wherein determining a C at position 61 indicates the SNP in the biological sample is from a chromosome from a canine having a small body size, and determining a T indicates the SNP in the biological sample is from a chromosome from a canine having a large body size; or the nucleotide at position 61 in SEQ ID NO:4, wherein determining an A at position 61 indicates the SNP in the biological sample is from a chromosome from a canine having a small body size, and determining a G indicates the SNP in the biological sample is from a chromosome from a canine having a large body size; or the nucleotide at position 61 in SEQ ID NO:10, wherein determining a G at position 61 indicates the SNP in the biological sample is from a chromosome from a canine having a small body size, and determining an A indicates the SNP in the biological sample is from a chromosome from a canine having a large body size; or the nucleotide at position 61 in SEQ ID NO:15, wherein determining a C at position 61 indicates the SNP in the biological sample is from a chromosome from a canine having a small body size, and determining an A indicates the SNP in the biological sample is from a chromosome from a canine having a large body size; or the nucleotide at position 61 in SEQ ID NO:18, wherein determining an A at position 61 indicates the SNP in the biological sample is from a chromosome from a canine having a small body size, and determining a C indicates the SNP in the biological sample is from a chromosome from a canine having a large body size; or determining a combination of said SNPs.

9. The method of claim 8, wherein the method comprises contacting the canine DNA with a first and second DNA probe wherein the first probe is specific for or can be extended to include a SNP that indicates the SNP in the sample is from a chromosome from a canine with a small body size, wherein the second probe is specific for or can be extended to include a SNP that indicates the SNP in the sample is from a chromosome from a canine with a large body size, and detecting hybridization or extension of only the first probe, or detecting hybridization or extension of only the second probe, or detecting hybridization or extension of the first and second probes.

10. The method of claim 9, wherein the first and second DNA probes are attached to a substrate.

11. The method of claim 10, wherein the first and second DNA probes are members of a plurality of distinct DNA probes in an array.

12. A method for determining in vitro in a biological sample comprising canine DNA a single nucleotide polymorphism (SNP) marker indicative of a phenotype of floppy ears, the method comprising testing the DNA to determine the nucleotide at position 61 in SEQ ID NO:3, wherein a G at position 61 indicates the SNP in the biological sample is from a chromosome from a canine that had floppy ears, and determining an A at position 61 indicates that the SNP in the biological sample is from a chromosome from a canine that did not have floppy ears.

13. The method of claim 12, wherein the method comprises contacting the canine DNA from the biological sample with a first and second DNA probe, wherein the first probe is specific for or can be extended to include a SNP that indicates the SNP in the sample is from a chromosome from a canine with the floppy ears, wherein the second probe is specific for or can be extended to include a SNP that indicates the SNP in the sample is from a chromosome from a canine with ears that are not floppy, and detecting hybridization or extension of only the first probe, or detecting hybridization or extension of only the second probe, or detecting hybridization or extension of the first and second probes.

14. The method of claim 13, wherein the first and second DNA probes are attached to a substrate.

15. An article of manufacture for use in determining a shedding phenotype for a canine, the article comprising a first and second DNA probe, wherein the first probe is specific for a segment of SEQ ID NO:1 that comprises nucleotide 61 that is a C, and wherein the second probe is specific for a segment of SEQ ID NO:1 that comprises nucleotide 61 that is T, wherein the first and second probes are attached to a substrate.

16. A method for determining that a biological sample comprises DNA that was obtained from a canine having a genome which comprises SEQ ID NO:1, wherein the nucleotide at position 61 is a T, or determining that the biological sample contains DNA that was obtained from a canine having a genome which comprises SEQ ID NO:1, wherein the nucleotide at position 61 is a C, the method comprising testing the DNA to determine the presence or absence of SEQ ID NO:1 and determining the nucleotide at position 61 in SEQ ID NO:1.


SEQUENCE LISTING (27 sequences)

SEQ ID NO: 1 - DNA, 121 bp, dog
  misc_feature (61)..(61): n is a, c, g, or t
ggaggaagaacggagcccagcacacgatgaagatgcccagcaacatggtcagggtgayggnccctctcatgctggccgtctgcctcacagagctgcagccacgcagagctgctatccgctt

SEQ ID NO: 2 - DNA, 121 bp, dog
  misc_feature (61)..(61): n is a, c, g, or t
ttgcacaaatcggtcctcgagcatgggaaatagattagcaggagaagctaaagtcagagcnggtaccccgggagcagtctgaagtgtcagggtctcagcccagcatctgaggtgggatgga

SEQ ID NO: 3 - DNA, 121 bp, dog
  misc_feature (61)..(61): n is a, c, g, or t
ttcacacccgcgggcggtggcacccaggggagcagcgggccgggtggcccggccgcggggngcagagccgagctgtaggcccggagccctggcccagggtcctcgtggcgcagctgggtcc

SEQ ID NO: 4 - DNA, 121 bp, dog
  misc_feature (61)..(61): n is a, c, g, or t
attagctccaagcaggagatacgctcctttctcttccaactacctactagtatttttatcnkgaggaggaaaaccagcccagaagccttctaagtatttaagtttaattattttaaatgat

SEQ ID NO: 5 - DNA, 121 bp, dog
  misc_feature (61)..(61): n is a, c, g, or t
ctgattcctgcccagtgctcttcgtgggatcctattatgcacaggagtcggtaagatgctngatagggatggattcctacaactgcccaatgatcagctctagagactctggagagctttg

SEQ ID NO: 6 - DNA, 121 bp, dog
  misc_feature (61)..(61): n is a, c, g, or t
acagagccgcacatgcgtcctctcgggctgtagatagtagccacgtaagacgctaataggntcatatctaattattccaggtgagacattgtaacagttctgggtgggaattccttctgtc

SEQ ID NO: 7 - DNA, 121 bp, dog
  misc_feature (61)..(61): n is a, c, g, or t
caccttagagtgggtactcaaccactgccgtataaacttccattccattaacattattacngagattactgaaataccaaattcatcattttatcctaagaacacattcagaaagaaatca

SEQ ID NO: 8 - DNA, 121 bp, dog
  misc_feature (61)..(61): n is a, c, g, or t
catttgttatatgtgtagtatgtacctgccaaactcttatatgaagccttcagacagttangtagggagcttgggaagtcagcaacagaaacctactctggccaacaagcaaaaatgggtg

SEQ ID NO: 9 - DNA, 121 bp, dog
  misc_feature (61)..(61): n is a, c, g, or t
accatggctcacttaaataaaattatctaagattgatatcataaaccaggtgtcattgccnggttcatttcggctgggaaatactaaatttgaaatatctttagtatggatcagcatggat

SEQ ID NO: 10 - DNA, 121 bp, dog
  misc_feature (61)..(61): n is a, c, g, or t
ctatagaagggaagtttaaagatctatgccttccctacccaccatatatctaccaccaccnccacccccactacagctaaactgaggatacaaacacgctgacaatgcaaatgaaggctga

SEQ ID NO: 11 - DNA, 121 bp, dog
  misc_feature (61)..(61): n is a, c, g, or t
agttatgactttagttatgacacatattgtttgttatgttacttagagaaaaatgaaaaantattcttgaaaagaggcccaataaagtcttctcaagggattggaaaagtgaaccaaacct

SEQ ID NO: 12 - DNA, 121 bp, dog
  misc_feature (61)..(61): n is a, c, g, or t
ttttgggcagagttgccaggagctctggatgcagctagcacttcttccatcattggaaatntggaaaatgtgtgggctttcacagggcctttggggacaaccccgtgtggttaggacccca

SEQ ID NO: 13 - DNA, 121 bp, dog
  misc_feature (61)..(61): n is a, c, g, or t
tgctctcgtctctccatctgaaagcaagtctctatagcatgactgcgtcttgctcctgatngggtctatactatgtcattgctccgtgaacacgagccctcaccctcccactgtactcaac

SEQ ID NO: 14 - DNA, 121 bp, dog
  misc_feature (61)..(61): n is a, c, g, or t
tacttttacaactccagtgttcccacctcgtgtcctaacaagaattgactgtgaatctctnatacacacagttttcagttcttgctttgcttcagaagaatcagcagtttgttgtgcaaaa

SEQ ID NO: 15 - DNA, 121 bp, dog
  misc_feature (61)..(61): n is a, c, g, or t
gtctttgaagttgtttaaggaactcatcagggaagagattttagcacatctatatatagtntttctcttgcctgttgatattaaagtcttttcaaaaatatgattcattagagtactgata

SEQ ID NO: 16 - DNA, 121 bp, dog
  misc_feature (61)..(61): n is a, c, g, or t
cagactctctgagatccagttttcttgtgttgacaaccaaagtactgcttggtatcgaggnctcattctaacaatatgaccctaaggattatctggatggttatgctacccattttttact

SEQ ID NO: 17 - DNA, 121 bp, dog
  misc_feature (61)..(61): n is a, c, g, or t
ataatttatctagttactcccccccactagtatgtctttatgtcctctcaaacaatacttntttcccattcctgtgtgaccacatgccatcaccatagtcaccatctcctcgatcaccaga

SEQ ID NO: 18 - DNA, 121 bp, dog
  misc_feature (61)..(61): n is a, c, g, or t
gaattatcaccccaagggcctatgagctcctgcaatagtcacagagcacctctacatggcnaaggatctttaaaggatttgatttattgagtacactacaattatggttattagtcactga

SEQ ID NO: 19 - DNA, 121 bp, dog
  misc_feature (61)..(61): n is a, c, g, or t
ttactcaacaagctcttaatgagtattgtgaatgactaaacccattcagattctacaaagnttggcctcatgaaagactttcacatgcctgaggtcatttatcctgcaaggtggaggatac

SEQ ID NO: 20 - DNA, 121 bp, dog
  misc_feature (61)..(61): n is a, c, g, or t
agagggcaccctagcagcacagctgaacctgaaatggatacaagccagggtgttctgagcntctccctgtaggagggggtgccctagtaggacagctctgactatgtcaaagtgggacata

SEQ ID NO: 21 - DNA, 121 bp, dog
  misc_feature (61)..(61): n is a, c, g, or t
tttttaaattgaactacttttccacttcaaatgaatatccacaatctacttttggggactngggtgctgggggcagtgtcagtacatgcatgaacacaggcggttaagagttcttaaaaac

SEQ ID NO: 22 - DNA, 121 bp, dog; identical to SEQ ID NO: 4
  misc_feature (61)..(61): n is a, c, g, or t

SEQ ID NO: 23 - DNA, 121 bp, dog
  misc_feature (61)..(61): n is a, c, g, or t
aatagtcggttcttagyatttcttaaaggaaaaaaagtagaggagatgtatttaaattagngcagagaagaatgatattaggcccatgggcctttcttgacaggccttctggcttgtgaca

SEQ ID NO: 24 - DNA, 121 bp, dog
  misc_feature (61)..(61): n is a, c, g, or t
gagattacctgagtgatgatgagcagggggatgaagcataaacctgagcccttgggctggngctaagtggtagagtctctgtatacaacttcagagaatcggctcttaacccacccacccc

SEQ ID NO: 25 - DNA, 50 bp, dog
cagctctgcgtggctgcagctctgtgaggcagacggccagcatgagaggg

SEQ ID NO: 26 - DNA, 121 bp, dog
  misc_feature (61)..(61): n is a, c, g, or t
aagcggatagcagctctgcgtggctgcagctctgtgaggcagacggccagcatgagagggnccrtcaccctgaccatgttgctgggcatcttcatcgtgtgctgggctccgttcttcctcc

SEQ ID NO: 27 - DNA, 121 bp, dog
  misc_feature (61)..(61): n is a, c, g, or t
gagagagaggtaacgggatcctgaagcagggctgaggaattagctatgcttgtgaggacanctgtcctcacagtctgattgcctcagcccgacactcaatcagcctggaggtgatgaggct